(19) United States
(12) Patent Application Publication
(10) Pub. No.: US 2010/0268271 A1
(43) Pub. Date: Oct. 21, 2010

(54) BRONCHIAL VALVE PLUG

(75) Inventor: Hideki Miyazawa, Toyama City (JP)

Correspondence Address: J. Rodman Steele;Novak Druce & Quigg LLP, 525 Okeechobee Blvd, Suite 1500, West Palm Beach, FL, 33401, US

(73) Assignee: NOVATECH SA, La Ciotat (FR)

(21) Appl. No.: 12/759,409

(22) Filed: Apr. 13, 2010

(30) Foreign Application Priority Data
Apr. 14, 2009 (FR) 0901812
Jan. 4, 2010 (FR) 1000009

(51) Int. Cl. A61B 17/03

(52) U.S. Cl. 606/213

(57) ABSTRACT

The bronchial plug (1) comprises a hollow body (2), the front side (4) of which is open and the back side (5) of which comprises at least one valve (6) being closed in the normal state thereof and adapted to be voluntarily or spontaneously opened so as to form a communication orifice. It further comprises gripping means (7) especially dedicated to a guide (10) adapted to position it in a bronchus (16), said gripping means (7) being arranged on the front side of said valve (6) and accessible through said valve (6).

### CROSS-REFERENCE TO RELATED APPLICATIONS

This application claims priority to French Patent Application 0901812, filed Apr. 14, 2009, and French Patent Application 1000009, filed Jan. 4, 2010, the contents of which are incorporated herein by reference.

### FIELD OF THE INVENTION

The present invention relates to a bronchial valve plug.

### BACKGROUND OF THE INVENTION

It is known that, to perform an embolization of a lung part affected with an air leakage due to either a pneumothorax or a bronchopleural fistula, bronchial plugs can be used, which are introduced through the endoscopic way into the segmentary or sub-segmentary bronchi of a patient. Such plugs are also used in the case of pulmonary emphysemas and for reducing the pulmonary volume. It is known from Patent Application FR 0207268 a bronchial plug which comprises a silicone hollow body with an approximately frustoconical shape, the front side (with respect to the introduction direction into the bronchi) of which is smaller than the back side. Moreover, the back side of the body is open, whereas the front side thereof is obstructed and carries, on the internal wall thereof, an internal projection adapted to be used as gripping means for a catheter adapted to position said plug in a bronchus. Thus, for such a positioning, the catheter having gripped said internal projection pushes the plug through the bronchi.

However, once positioned, the bronchial plug obstructs totally the bronchus in which it has been positioned. So, in case of an overpressure within the downstream part of the bronchus beyond the front side of the plug, it is frequent that hyperinflation phenomena appear in emphysematous patients, the air accumulated in the downstream part of the bronchus being unable to be discharged up to the upstream part thereof.

In order to remedy such disadvantage, it is known to use a bronchial valve plug, which comprises a hollow body made of elastic silicone material, the front side (again defined with respect to the introduction direction into the bronchi) of which is open and the back side comprises a unidirectional valve being closed in the normal state thereof and adapted to be voluntarily or spontaneously opened so as to form a communication orifice. This latter of a slot type is provided with two spontaneously contiguous edges as a flat lip in a duck-billed way.

So, when it has been introduced into a bronchus, the bronchial plug can, in case of an overpressure in the downstream part of the bronchus beyond the front side thereof, cancel at least partially such overpressure through the valve in the back side thereof, the spontaneously jointed edges of which are opened elastically in order to let the pressurized air be vented outside. Such a bronchial plug has thus as an object to replace (at least in a first intent) the pulmonary volume reduction through a surgical way.

The positioning of such a plug into a bronchus is generally performed through a guide provided at its distal end with a flexible clip adapted to grip the edges being shaped as a flat lip of the communication orifice in the back side. Once the edges being gripped, the guide pushes the plus into the bronchi.

However, as it is pushed with the back side thereof, the bronchial plug tends upon its positioning to be located crosswise in said bronchi, what makes the operation difficult, long and delicate.

Moreover, as the plug is made of a flexible silicone material, when it grips the edges of the communication orifice in the back side, the guide clip crushes the flexible material of said edges which frequently only reassumes its initial shape when the clip pressure is released. The efficiency of the valve is then altered.

### SUMMARY OF THE INVENTION

The present invention has for an object to remedy such disadvantages.

To this end, according to the invention, the bronchial plug comprising a hollow body, the front side of which is open and the back side of which comprises at least a valve being closed in the normal state thereof et adapted to be voluntarily or spontaneously opened so as to form a communication orifice, is remarkable in that said plug comprises gripping means especially dedicated to a guide adapted to position it in a bronchus, and in that said gripping means are arranged on the front side of said valve and accessible through said valve.

So, through the invention, as the body of the bronchial plug is hollow and the back side of this latter comprises a valve, the positioning guide can enter within said body through such valve so as to reach the gripping means, in front of said valve. It results that the bronchial plug is not any more pushed by the back upon positioning and it does not any more tend to be located crosswise, as being threaded on the distal end of the guide.

In order to further improve the positioning conditions for the bronchial plugs, it is advantageous that said gripping means are arranged in the vicinity of said front side of said body.

So, during the introduction of the bronchial plug according to the invention in a bronchus, the plug holding with the distal end of the guide is optimum and the pushing centre exerted by the guide on the plug is the most forward possible.

Advantageously, said gripping means are formed as a handle, the gripping of which is easy. Moreover, the handle shape does not disturb or very little the operation of the bronchial plug, the opening of the front side staying released.

Moreover, the longitudinal ends of said handle are preferably built in (for example through sticking) on the internal wall of said body. Of course, optionally, it can be consider that the handle and the hollow body of the valve form only one material block.

Further, in order to make positioning of the bronchial plug easier in a bronchus, it is advantageous that at least the front part of said body has a substantially frustoconical shape.

Preferably, said communication orifice of slot type comprises two edges, as a flat lip, that are contiguous spontaneously in the normal state of said valve. So, the gripping means avoid for the edges of the communication orifice to be gripped by the guide, which could alter them.

Of course, it can be considered to use any other type of a communication orifice, the edges of which would be spontaneously contiguous.

Moreover, in order to further improve the introduction of the bronchial plug into the bronchi, the contour of said front side comprises advantageously notches. Such notches enable to reversibly reduce the section for said front side so as to make the introduction of the plug easier into bronchi of a lower section than the hollow body.

Preferably, said hollow body and said gripping means are made in silicone or a similar material. However, optionally, the hollow body and the gripping means can be made each from a different material.

Moreover, the hollow body and the gripping means can also be formed with a flexible mesh structure, for example a metallic one, covered by a flexible watertight coating, for example in silicone or any similar material.

In order to provide the right holding for the plug of the invention in a bronchus, said hollow body comprises on the external side wall thereof, holding projecting means, for example external pins adapted to be abutted against the internal wall of said bronchus.

### BRIEF DESCRIPTION OF THE DRAWINGS

The figures of the accompanying drawing will make clear how the invention can be implemented. On the figures, identical numbers refer to similar members.

FIGS. 1 and 2 are two distinct elevation views of a bronchial valve plug according to the present invention.

FIG. 3 is an axial section along the line III-III of FIG. 1

FIG. 4 is a view of the back of the bronchial plug of FIG. 1, the valve of the back side thereof being closed.

FIG. 5 is a view similar to FIG. 4, the valve of the bronchial plug being opened.

FIGS. 6A to 6C show the different positioning steps of the bronchial plug of FIG. 1 in a bronchus through a clip guide.

### DETAILED DESCRIPTION

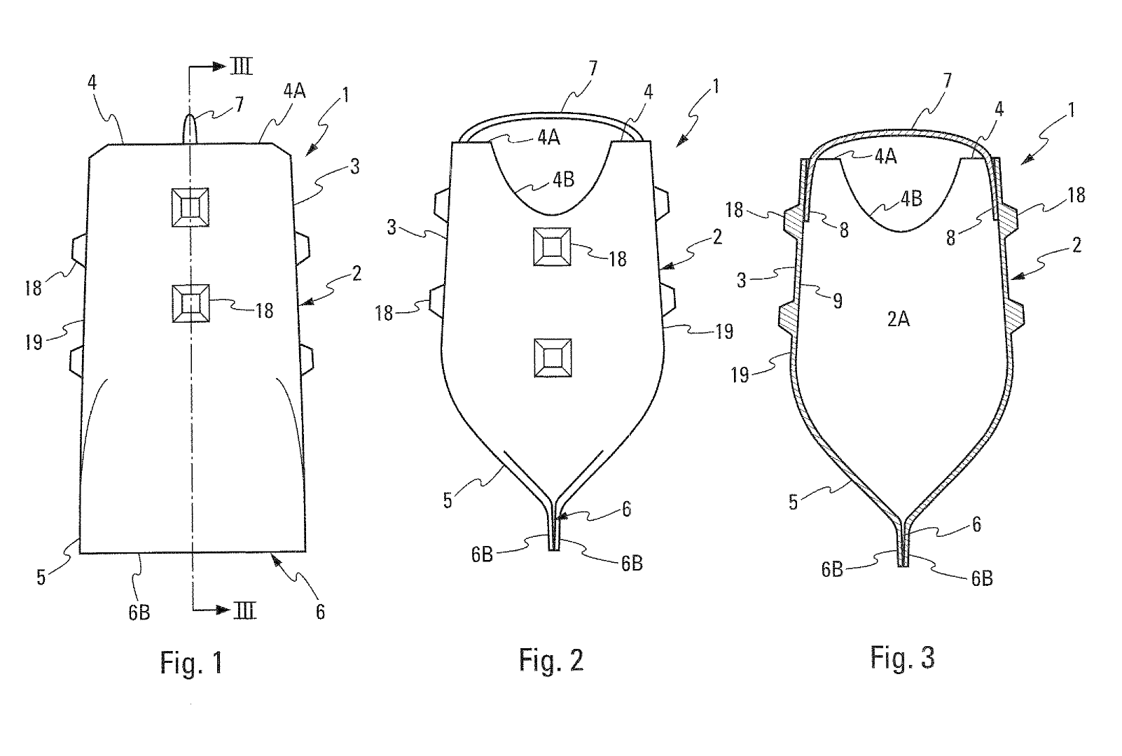

On FIGS. 1 to 3, a bronchial valve plug 1 according to the invention has been shown. The plug 1 comprises a hollow body 2 in silicone or any similar material, comprising an internal cavity 2A. Other materials can be used; in particular, the hollow body 2 can be made (at least partially) in titanium.

In the shown embodiment, the front side 3 of the hollow body 2 has an at least approximately frustoconical shape. Obviously, different shapes can be provided for the body 2; in particular, the body 2 can be totally in a cylindrical shape, totally in a frustoconical shape or even comprise a portion in a cylindrical shape and a portion in a frustoconical shape located in front of such cylindrical portion.

The bronchial plug 1 comprises a front side 4 (with respect to the introduction direction into the bronchi) being opened and a back side 5 which comprises a valve 6 being closed in the normal state thereof et adapted to be voluntarily or spontaneously opened so as to form a communication orifice 6A. The terms front and back are frequently designated, in the field of the bronchial plus, by distal and proximal, respectively.

The communication orifice 6A of the slot type comprises edges 6B, as a flat lip, that are held spontaneously contiguous in the normal state of said valve 6.

According to a non shown embodiment, the valve 6 comprises, at the base of the lips 6B, reinforcing means enabling to avoid an inopportune opening of the lips 6B once they have been positioned.

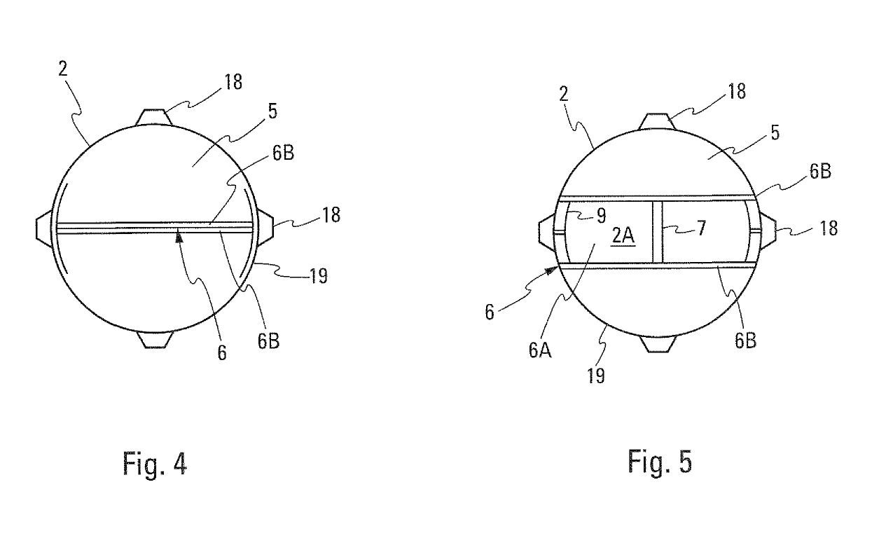

So, as shown on FIGS. 4 and 5, once the plug 1 positioned in a bronchus, when the fluid pressure (generally air) in the downstream part of the bronchus beyond the front side 4 is at most the same as the pressure in the upstream part of the bronchus this side of the back side 5, the valve 6 stays closed (FIG. 4), the edges 6B of the communication orifice 6A being held spontaneously contiguous through the elasticity in the wall constituent material of the body 2.

On the contrary, when the fluid pressure in the downstream part of the bronchus is higher than the pressure in the upstream part of the bronchus, the edges 6B of the orifice 6A can be moved away from each other elastically while fluid is escaping (FIG. 5). The valve 6 is then opened.

As shown on FIGS. 2 and 3, the contour 4A of the front side 4 comprises two notches 4B being facing, which enable to reduce reversibly the section of the front side 4 so as to make the positioning of the plug 1 easier in the bronchus of small diameter.

According to the invention, as shown on FIGS. 1 to 3, the bronchial plug 1 further comprises a curved guiding handle 7, for example in silicone or a similar material, arranged in front of said plug 1. As shown on FIG. 3, the longitudinal ends 8 of the guiding handle 7 are built in (for example through sticking) on the internal wall 9 of the hollow body 2, in the vicinity of the front side 7.

It is quite obvious that the longitudinal ends of the handle 7 could also be arranged in any desired other way (for example on the contour of the front side of the bronchial plug).

Moreover, it is also possible to make integrally with the same material the handle 7 and the hollow body 2 of the plug 1.

It is also possible to make the hollow body 2 and the handle 7 in two different materials, for example the hollow body 2 in titanium and the handle 7 in silicone.

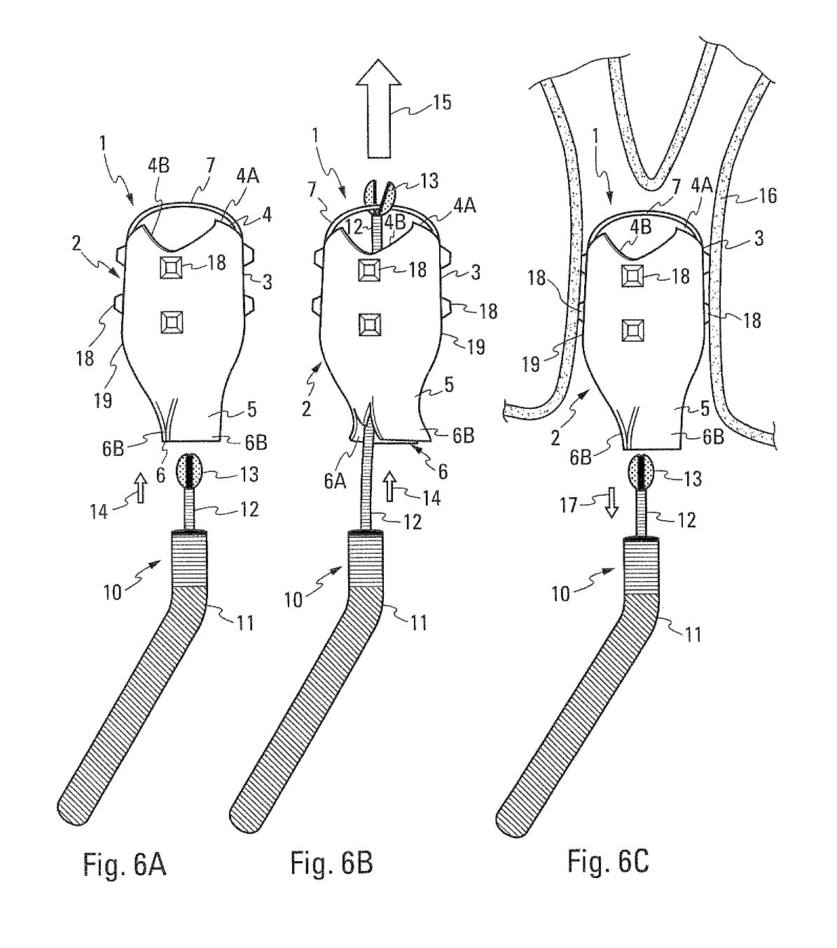

On FIGS. 6A to 6C, there is described the positioning of the bronchial plug 1 according to the invention through a guide 10. This latter comprises a catheter 11 (for example a fiberscope) in which there is a guiding rod 12 which can be moved provided, at the distal end thereof, with a flexible clip 13, the opening and the closure of this latter being ordered by an operator.

First of all, as shown on FIGS. 6A and 6B, the guiding rod 12 is extended out of the catheter 11 (the movement towards the front of the rod 12 is marked by the arrow 14) so as to enable her to be introduced in the cavity 2A of the hollow body 2 after crossing the valve 6 of the back side 5 of the plug 1 and reaching the guiding handle 7. Then, the flexible clip 13 can be opened so as to grip the guiding handle 7.

When the guiding handle 7 has been gripped by the clip 13, the extension of the guiding rod 12 can go on so as to push forwardly the plug 1, as shown by the arrow 15. It results that the pushing centre for the guiding rod 12 of the guide 10 is located in front of the bronchial plug 1 so that the follow up of the advance path by the plug 1 is optimum.

As shown on FIG. 6C, when the bronchial plug 1 has been correctly positioned in a bronchus 16, the guiding rod 12 is put back into the catheter 11 (the movement towards the back of the rod 12 is marked by the arrow 17) so as to extract the plug 1.

Moreover, projecting pins (for example, frustoconical) carried by the external side wall 19 of the bronchial plug 1, enable to hold said plug 1 in position within the bronchus 16 (FIG. 6C). Any other adapted holding means 1 in position can be provided, in particular any structure or member projecting out of the external surface of the side wall 19 of the body 2 of the plug 1, for example one or more circumferential rings.

1. Bronchial plug comprising a hollow body (2), the front side (4) of which is open and the back side (5) of which comprises at least a valve (6) being closed in the normal state thereof and adapted to be voluntarily or spontaneously opened so as to form a communication orifice (6A), wherein said plug (1) comprises gripping means (7) especially dedicated to a guide (10) adapted to position it in a bronchus (16), and in that said gripping means (7) are arranged on the front side of said valve (6) and accessible through said valve (6).

2. Bronchial plug according to claim 1, wherein said gripping means (10) are arranged in the vicinity of said front side (4) of said body (2).

3. Bronchial plug according to claim 1, wherein said gripping means (7) are formed as a handle.

4. Bronchial plug according to claim 3, wherein the longitudinal ends (8) of said handle (7) are preferably built in on the internal wall (9) of said body (2).

5. Bronchial plug according to claim 1, wherein at least the front part (3) of said body (2) has a substantially frustoconical shape.

6. Bronchial plug according to claim 1, wherein said communication orifice (6A) of slot type comprises two edges (6B) as a flat lip, that are contiguous spontaneously in the normal state of said valve (6).

7. Bronchial plug according to claim 1, wherein the contour (4A) of said front side (4) comprises notches (4B).

8. Bronchial plug according to any claim 1, wherein said hollow body (2) and said gripping means (7) comprise silicone.

9. Bronchial plug according to claim 1, wherein said hollow body (2) comprises on the external side wall (19) thereof, holding projecting means (18), for example external pins (19) of circular or rhombus section.
